(19) United States
(12) Patent Application Publication
(10) Pub. No.: US 2020/0281223 A1
(43) Pub. Date: Sep. 10, 2020

(54) PURIFICATION METHOD

(71) Applicant: The Walter and Eliza Hall Institute of Medical Research, Victoria (AU)

(72) Inventor: Gregory John Tanner, Victoria (AU)

(73) Assignee: The Walter and Eliza Hall Institute of Medical Research, Victoria (AU)

(21) Appl. No.: 16/291,326

(22) Filed: Mar. 4, 2019

(51) Int. Cl. A23J 1/12; C07K 1/14; C07K 1/30; C07K 1/34; C07K 14/425

(52) U.S. Cl. CPC A23J 1/12; C07K 1/145; C07K 14/425; C07K 1/34; C07K 1/30

(57) ABSTRACT

The present disclosure is directed to methods for purifying prolamin proteins from a cereal flour containing such proteins. More particularly, the present disclosure provides a rapid and cost effective method for the purification of avenin proteins from oat flour, gluten proteins from wheat flour, secalin proteins from rye flour, hordein proteins from barley flour, zein proteins from maize flour or kafirin proteins from sorghum flour.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### FIELD OF THE DISCLOSURE

The present disclosure is directed to methods for purifying prolamin proteins from a cereal flour containing such proteins.

### BACKGROUND OF THE DISCLOSURE

Coeliac disease (CD) is an inflammatory disorder of the small intestine which is precipitated in genetically susceptible individuals by gluten resulting in damage to the small bowel in which the villi become inflamed and flattened (villous atrophy). Consequently, the surface area of the bowel available for nutrient absorption is reduced resulting in various gastrointestinal and malabsorptive symptoms. The disorder occurs in approximately 1% of most populations (Fasano B et al., (2003). Archives of Internal Medicine, 163(3), 286-292), when sensitive subjects carrying DQ2(8) restricted T-cells react inappropriately to key amino-acid epitopes present in dietary gluten (Anderson R P et al., (2000) Nature Medicine, 6, 337-342).

Massive clonal expansion of specific CD4+ T-cells takes place—reacting as if the body were facing an infection by invading microflora—ultimately leading to the destruction of intestinal microvilli. The limited number of key amino acids involved in the coeliac response are recognised by circulating T-cells approximately 6 days after dietary challenge (Anderson, R. P., (2005). Gut, 54, 1217-1223) and have been comprehensively mapped in all published gluten protein sequences (Beissbarth, T et al. (2005) Bioinformatics, 21, I29-I37; Tye-Din, J. A et al., (2010) Science Translational Medicine, 2(41), 41 ra51). From this sequence data, a vaccine approach for coeliacs is being developed (Goel G et al., (2017) The Lancet Gastroenterology & Hepatology, 2(7), 479-493). The epitope sequence data also supports the well-known contention that coeliacs must maintain a lifelong avoidance of wheat, barley and rye.

Alcohol soluble fractions of wheat (gliadin), oats (avenin), barley (hordein), and rye (secalin), are thought to active the disease process and are referred to as prolamins. The alcohol soluble fractions of wheat, barley and rye are referred to as glutens and for oats are referred to avenins. The prolamin content in wheat, barley and rye constitute about 40% of the protein whereas it comprises about 15% in oats.

With regard to oats, the safety of oat consumption by coeliacs is still subject to controversy. Unlike gluten which contains gliadin, oats contain the counterpart avenin. Like all other cereal grain, oats belong to the Poacease family of which Avena sativa is the most important crop. Oats are the sixth most significant cereal cop in the world, with production exceeding 24 million tonnes annually. The health effects of oats have been primarily attributed to the highly viscous β-glucan fraction which has the ability to lower blood cholesterol and the intestinal absorption of glucose.

Although most patients with CD seem to be able to tolerate oats, a considerable number of cases of intolerance to pure oats have been identified (Lundin K E et al. (2003) Gut Nov; 52(11):1649-52) suggesting that oats should be excluded when prescribing a gluten-free diet.

Evidence from T-cell epitopes provoked in response to a dietary oat challenge suggest that 8% of coeliacs raise a genuine T-cell mediated response to amino-acids in oat prolamins (avenins) and therefore react to oats (Hardy M Y et al. (2015) Journal of Autoimmunity, 56, 56-65). Conflicting claims on the safety of oat consumption from feeding trails by coeliacs have been published and the question has not been resolved.

There are two problems with oat feeding studies. Firstly, oats used in some feeding studies may have been inadvertently contaminated with trace wheat or barely grains—wheat, barley and oats are grown in the same areas and often harvested and transported with the same machinery. In countries such as Europe, Canada and the United States, it has been found that up to 80% of samples had some level of contamination and the primary cereal was barley. In Australia, ten (10%) percent contamination may be encountered (Bekes, F. Director FBFD Pty Ltd, personal communication) Europe, UK, Canada and USA have adopted the Codex standard for gluten free foods while Australia does not accept the Codex standard. The presence of a single grain of wheat in 200 gm of oats can result in the wheat gluten level of greater than 100 ppm; well above the 20 ppm level set in most legislations as the upper limit for gluten-free food status. Failure to provide harvesting, transport and milling facilities dedicated to oats, may easily result in serious inadvertent contamination by wheat grains.

Contamination of oat crops by rye grass is also possible—rye grass is a common weed in cereal crops and difficult to remove by air-driven seed cleaning techniques as it has similar density properties as cereals. Rye grass contains many antigenic gluten-like proteins, with dominant proteins of 30 and 50 kDa that are identified by anti-gluten antibodies (Colgrave M L et al. (2015) Journal of Proteome Research, 14(6), 2659-2668). It is likely that these proteins may provoke a coeliac response and should be avoided in gluten-free cereal crops.

The second difficulty with oat feeding studies may arise from supplying insufficient avenin to provoke a response in the less sensitive coeliacs. The low rate of response found in (Hardy, M Y et al., (2015) Journal of Autoimmunity, 56, 56-65) suggests that common dietary challenges of 100 g oats per day was insufficient to raise a coeliac response in many of these subjects. The response of coeliacs to barley gluten (hordein) varies by at least a hundred fold—ie some coeliacs respond less strongly to gluten and require higher concentrations to mount a response. Oat avenins are present at approximately 1% of the grain protein—much lower than wheat (70%) or barley (50%) and unlike wheat bread or barley which require 1/50th slice of bread or a several grains of barley to provoke a coeliac response—an oat challenge may require the consumption of more than 300 grams of oats per day—a difficult task for a volunteer.

Present methods for extracting glutens and avenins from cereals require large amounts of water and the use of heat. Traditional methods for isolating gluten from gluten containing cereals such as flour (usually referred to as ‘wet processing”) require the use of water to wash out the water soluble products. In general, protein from starch separators use large amounts of water as the fractionation fluid, remove water-soluble protein and discharge large amounts of dilute protein-bearing aqueous waste. Separators and the associated dryers use excessive energy, are capital intensive and subject the gluten to loss of end-use functionality.

Thus there exists a need for methods to isolate food grade avenins, in sufficient quantity and purity and uncontaminated by other gluten containing grains for use in feed trials for subjects who are coeliac. There is also a need in the art for methods for the purification of prolamin proteins, for example gluten from wheat, hordein from barley, secalins from rye that are commercially and environmentally better than existing prior art methods.

### SUMMARY OF THE DISCLOSURE

Fortuitously, the inventors discovered that chilling ethanol extracted avenin proteins provides selective precipitation of the avenin protein fraction and separation from the starch fraction, producing a milky white precipitate comprising substantially purified avenin proteins that can be harvested for various uses. This method was also found to be useful for the separation of other prolamin proteins from their source, for example gluten from wheat.

The present disclosure is directed to a novel method for isolating and purifying alcohol soluble proteins (e.g. prolamins) from cereal grains containing such proteins. Advantageously, the purified proteins are produced in high purity and are of a food grade standard suitable for human consumption.

In a first aspect, the present disclosure provides a method for purifying alcohol soluble proteins (e.g. alcohol soluble prolamins) from a cereal flour comprising said proteins, wherein the method is performed without hydrating the cereal flour with water.

Traditional purification methods for harvesting gluten proteins use a water wash of the flour to remove most of the starch followed by drying of the wet residue. Protein fractions obtained by this method produce a sticky, glutinous, cohesive mass that dries slowly. Additionally, and manipulating and drying industrial quantities using gentle heat to avoid damaging the baking properties of the gluten is difficult and slow. Advantageously, the method produces alcohol soluble protein fractions that are equivalent, if not superior in yield and protein concentration to fractions produced by water washing methods, while avoiding the disadvantages of the water washing methods. Thus, provided is a method for purifying prolamin proteins from a cereal flour comprising said proteins, wherein the method is performed without hydrating the cereal flour with water.

The method of the present invention is suitable for the purification of any prolamin proteins from their source. Such prolamin proteins include gluten proteins from wheat, secalin proteins from rye, hordein proteins from barley, avenin proteins from oats, zein proteins from maize and kafirin proteins from sorghum. Additionally, the method is suitable for the isolation of prolamin proteins from mixtures of the above sources, for example a mixture of wheat and rye. In a particular example, the prolamin proteins are avenin proteins.

Prolamins from any cereal flour containing same can be purified according to the disclosed methods. For example, the cereal flour may be derived from a cereal grain selected from the group consisting of wheat, barley, rye, maize, rice, sorghum or oats. In another example, the cereal flour is selected from the group consisting of wheat flour, barley flour, rye flour, maize flour, rice flour, corn flour, sorghum flour and oat flour. In another example, the cereal flour is oat flour. In another example, the cereal flour is wheat flour.

In one embodiment, the method comprises:

(i) mixing the cereal flour with an organic solvent in an amount sufficient to substantially wet the flour and form an admixture with the flour;

(ii) chilling a supernatant for a time sufficient for the prolamin proteins to precipitate, wherein the supernatant is obtained by physically separating the cereal flour and solvent admixture of step (i); and

(iii) harvesting the precipitated prolamin proteins from the chilled supernatant of step (ii).

In certain examples the organic solvent is selected from ethyl alcohol (ethanol), isopropyl alcohol, methyl alcohol, acetone, propanol, dimethylsulfoxide (DMSO), or dimethylformamide (DMF). In a particular example, the alcohol is ethyl alcohol (ethanol).

In some examples, the organic solvent is provided at a concentration range of about 40-70% v/v prior to admixing with the flour. In a particular example the organic solvent is provided at a concentration range of about 50% v/v prior to admixing with the flour.

In certain examples, the cereal flour and organic solvent are admixed in a ratio of about 1:1.5 to 1:4. In further examples the cereal flour and organic solvent are admixed in a ratio of about 1:1.5 to 1:2, more preferably 1:1.5. Persons skilled in the art will know to adjust the amount of organic solvent so that flour is suitably wetted and the admixture can be readily stirred without too much resistance.

Mixing of the cereal flour and organic solvent should be performed for a time sufficient to substantially extract the prolamin proteins from the cereal flour. The period of mixing should allow for at least 50%, at least 60%, at least 70%, at least 80% or at least 90% of the prolamin proteins to be extracted from the cereal flour. In one example the period of mixing time allows for 95% or greater prolamin proteins to be extracted from the cereal flour.

Depending on the flour content, the extraction period may last anywhere from between 10-30 mins on a laboratory scale to about 1 to 24 hours on a commercial scale. In certain examples, the extraction period is performed overnight (about 8 to 12 hours) for convenience.

In certain examples, the mixing is carried out at ambient temperature.

The mixing process may be continuous or intermittent. The cereal flour and organic solvent may be mixed by stirring or shaking. Examples of suitable machines include vortex machine, blender, or magnetic stirrer. For larger scale preparations, commercial scale vertical mixers such as a Hobart mixer can be used.

With regard to step (ii), the supernatant fraction of the cereal flour and solvent admixture can be obtained by any suitable physical separation means known in the art. Such methods include filtration, centrifugation, decanting or settling. In certain examples, the supernatant fraction is obtained by centrifugation of the admixture of step (i). Persons skilled in the art will be able to determine by test runs and trial and error, the appropriate parameters for centrifugation which achieves a firm pellet. Various factors affect centrifugation including density, temperature/viscosity, distance of particle displacement and rotational speed. By way of non-limiting example, the inventors have found that for a sample volume of 500-750 mL a centrifugation speed of about 500-800×g is sufficient for a period of about 5-10 mins. In preferred examples, the centrifugation is carried out at ambient temperature. In one example the centrifugation process results in the production of a pellet comprising the flour and starch.

In certain examples, the supernatant is chilled for a sufficient time to allow substantial precipitation of the prolamin proteins within the flour. In certain examples, at least 70%, at least 80%, at least 90%, greater than 95% or greater than 98% of the prolamin proteins are precipitated from the supernatant. In certain examples, precipitation of the prolamin proteins will be evident as a milky white suspension.

The chilling time may range from about 10 mins to 30 mins or longer. Chilling may be performed overnight or for a period of one to several hours. The person skilled in the art will be able to determine a suitable chilling time depending on the volume of the supernatant.

In certain examples, the chilling temperature is between about 4 and 15° C. In other examples, the chilling temperature is between about 4 and 10° C., more preferably between about 4 and 6° C.

Harvesting of the precipitated prolamin proteins can be achieved by methods known in the art. For example, the prolamin protein fraction may simply be allowed to settle and can be recovered by decanting the supernatant. Collection efficiency and concentration of the prolamin proteins can be achieved by centrifugation of the chilled supernatant at a chilled temperature, for example, between about 4 and 12° C. The present inventors have found that precipitation of the prolamin proteins can be reversed and re-dissolving of the proteins occurs as the temperature warms up beyond 15-18° C.

In certain examples, multiple rounds of resuspension and centrifugation of the precipitated prolamin proteins may be performed wherein the pellet is resuspended in the solvent (e.g. 50% v/v ethyl alcohol). In certain examples, pre-chilled solvent is used for resuspending the pellet.

Depending on the sample volume the skilled person would be able to determine the appropriate centrifugation speed and time. In certain examples, the centrifugation speed is about 3,000 to 5,000×g. In certain examples, the samples are centrifuged for a period of about 10-15 mins. In further examples, the centrifugation is carried out at a temperature between about 4 and 12° C. Preferably, the centrifugation should be carried out for a sufficient time and speed to obtain a clear honey-like liquid at the bottom of the sample container. Thus, in certain examples, harvesting the precipitated prolamin proteins comprises concentrating the proteins by centrifugation to produce purified prolamin proteins.

The sample container may be of any suitable size in which to perform the centrifugation. For example, the sample container may vary in size from a 50 mL tube to a 500 mL tube or larger for industrial applications.

In some examples, the final pellet is resuspended in a minimal volume of water or solvent for storage. In one example, the pellet is resuspended in an alcohol concentration which is sufficient to prevent microbiological growth. In one example, the pellet is resuspended in 10% v/v alcohol solvent. By “minimal volume” it is meant a volume of water to solvent which is sufficient to form a fine suspension of the prolamin proteins. In certain examples, a minimal volume is a 1:1 ratio, for example 10 g pelleted protein per 10 mL of water or solvent. In certain examples, the final resuspended pellet comprising purified prolamin proteins is stored chilled. In other examples, the final resuspended pellet comprising purified prolamin proteins is stored frozen.

In certain examples, the final resuspended pellet comprising purified prolamin proteins is processed to a powdered form.

Accordingly, the method may further comprise producing a powder of purified prolamin proteins by:

(i) optionally homogenising the purified prolamin proteins; and

(ii) evaporating the water or alcohol.

In certain examples, homogenising is achieved by mixing, blending or vortexing.

Evaporation of the alcohol or water may be obtained by various methods known in the art, including freeze-drying, evaporation in air, or drying with heat. In one example, the evaporation is achieved by freeze drying the precipitated prolamin proteins following homogenisation. In another example, the evaporation is achieved by ring (spray) drying which is conventional in the art.

In one example, the powder may be stored chilled, for example at a temperature between about 4 and 15° C. In another example, the powder is stored in a freezer. In a further example, the powder is stored at room temperature. In certain examples a desiccant or humectant may be included with the powder so that the powder remains dry.

In another embodiment, the method of the disclosure may further comprise preparing a cereal flour from a cereal containing prolamin proteins. Methods of milling cereals to form a flour are known in the art and may incorporate the use of grinders and/or mesh filters. In certain examples, the flour may be subjected to further milling, for example such as grinding in a blender to produce a finer powder. In certain examples, the average particulate size of the flour is from about 100-400 microns, more preferably from about 150-250 microns.

In a second aspect, the disclosure provides a composition comprising substantially purified prolamin proteins prepared by the method according to the first aspect of the disclosure.

In a third aspect, the disclosure provides a composition comprising prolamin proteins having a purity greater than 90%. In one example, the purity is about 92%, 93%, 94%, 95% 96% or 97%. In another example, the purity is greater than 97%. In a further examples, the purity is about 91-94%, preferably about 93±0.5%. In another example, the purified prolamin proteins are selected from the group consisting of gluten, secalin, hordein, avenin, zein and kafirin.

In one example, the recovery of purified prolamin proteins from the cereal flour is greater than 60%, greater than 70%, greater than 80%, greater than 90%, or greater than 95%. In another example, the recovery is 97%, 98%, 99% or 100%.

In a third aspect, the present disclosure provides a food product or additive comprising substantially pure prolamin proteins harvested from a cereal flour.

In a fourth aspect, the present disclosure provides a food product or additive comprising substantially pure avenin proteins from oat flour.

In a fifth aspect, the present disclosure provides a food product or additive comprising substantially pure gluten proteins from wheat flour.

In a sixth aspect, the present disclosure provides a food product or additive comprising substantially pure secalin proteins from rye flour.

In a seventh aspect, the present disclosure provides a food product or additive comprising substantially pure hordein proteins from barley flour.

In an eighth aspect, the present disclosure provides a food product or additive comprising substantially pure zein proteins from maize flour.

In a ninth aspect, the present disclosure provides a food product or additive comprising substantially pure kafirin proteins from sorghum flour.

In certain examples, the food product or additive is obtained by the method according to the first aspect.

In one example, the substantially pure prolamin proteins are provided in powdered form.

In a tenth aspect, the present disclosure provides for the use of substantially pure prolamin proteins prepared according the disclosed methods for improving dough strength and elasticity. In one example, the prolamin protein is selected from the group consisting of gluten, secalin, hordein, avenin, zein and kafirin.

### DESCRIPTION OF THE FIGURES

FIG. 1 provides a flow diagram of the purification method of the present disclosure.

FIG. 2 shows the effect of solvent concentration on avenin precipitation. Adding water or EtOH precipitated avenin, however the water induced precipitate resulted in a colloidal suspension that could not be conveniently pelleted.

FIG. 3 shows small scale avenin precipitation by chilling at 4° C. A precipitate formed as the 50% ethanol (EtOH) extract cooled below 15° C. and appeared to be complete at 10° C. This could be reversed 10 times, by warming to 20° C. and cooling again to 4° C.

FIG. 4 shows purity of small scale avenin precipitation at 4° C., or with increasing ethanol (EtOH) concentration. Total proteins are shown in A-stained with Coomassie Blue. Avenin proteins are shown in B after Western Blotting with Sigma antigliadin-HRP labelled antibody. This commercial antibody has previously been shown to detect all gluten protein families (Colgrave, M. L., et al., (2015) Journal of Proteome Research, 14(6), 2659-2668). Each lane contains equal protein load of (A) 20 μg—protein gel, and (B) 2 μg—western blot. A and B are calibrated with 10 kDa pre-stained standards (Invitrogen), which were calibrated with 10 kDa unstained standards. Avenin bands are numbered 1-6. Total protein was stained with Coomassie Blue.

FIG. 5 shows the effect of extraction on avenin yield. Maximum freeze-dried avenin yield was observed after a two-day extraction. Oat flour was extracted with 50% IPA for the indicated time and protein content of the supernatant measured.

FIG. 6 shows the recovery and purity of two day, 500 g extraction. Total protein and specific avenin content of fractions by protein Urea-SDS-PAGE (A) and western blot (B). Lanes are loaded with constant volume relative to S1 (supernatant). Avenin bands are indicated 1-6. Twenty (FIG. 6A, 20 μg) or four (FIG. 6B, 4 μg) micrograms of the final freeze dried avenin are shown in right hand lanes. Fractions are as described in the method “five hundred pram oat extract” below. The majority of the avenin is recovered in the final combined supernatant (S4).

FIG. 7 shows two (2) grams of freeze-dried avenin powder harvested from 500 g oat flour.

FIG. 8 shows the purity, molecular weight, and yield of purified avenin. The total protein gel by urea-SDS-PAGE (A), and specific avenin proteins by western blot (B) of the combined 50% EtOH extract (S1+2), the 4° C. supernatant (S4) of a single avenin preps, and two avenin preps (Prep1, Prep2) are shown calibrated against prestained 10 kDa ladder (PS). The molecular weights of the prestained ladder are inflated by bound dye molecules and were calibrated against an unstained 10 kDa protein ladder (Invitrogen) (FIG. 7, US) which is a recombinant protein ladder of accurate size. Note: The prestained 10 kDa standard does not bind to the western blot.

FIG. 9 shows the purity of avenin isolation from 200 kg avenin preparations by SDS-PAGE protein gel (A) or western blot (B). Two batches of oats consisting of two hundred kilograms of oat flour was successively purified to yield two lots of purified avenin (Prep 1) and (Prep 2) are shown calibrated against prestained 10 kDa ladder (PM). Avenin was dissolved in 8M urea, 1% DTT, 20 mM TEA (pH6), protein measured and 1, 2, 4 or 5 ug loaded per lane as shown.

FIG. 10 shows a photo of 0.9 kg freeze dried avenin powder.

FIG. 11 shows chill induced precipitation of gluten proteins. Gluten proteins were isolated from wheat, barley and oats by chill precipitation (WCP, BCP, OCP respectively, Chill ppted) and compared to gluten proteins freshly isolated by extraction of wheat, barley and oats in 50% IPA, 1% DTT (W1, W2, B1, B2, O1 and O2 respectively, 50% IPA, 1% DTT) by SDS-PAGE (A; 20 μg per lane), or western blot (B; 2 μg protein per lane). Corresponding protein bands calibrated against pre-stained standards (Benchmark, Invitrogen) are numbered 1-28 in both images. The pre-stained standards were themselves calibrated against Benchmark unstained standards which have accurately designated molecular weights. In each case gluten proteins by definition extracted by 50% ethanol are present in the corresponding chill precipitated fractions. This was observed for Coomassie stained protein bands (protein gel) and gluten specific proteins by Western Blot (Western Blot).

FIG. 12 shows the discrete bands of flour and starch formed in the pellet following centrifugation of the cereal flour and ethyl alcohol admixture. The ethyl alcohol, oil and gluten are in the supernatant fraction.

### DETAILED DESCRIPTION

### Term and Definitions

Reference to the singular forms “a”, “an” and “the” is also understood to imply the inclusion of plural forms unless the context dictates otherwise.

Throughout this specification, unless the context requires otherwise, the word “comprise” or variations such as “comprising” or “comprises” will be understood to imply the inclusion of a stated step or element or integer or group of steps or elements or integers but not the exclusion of any other step or element or integer or group of steps or elements or integers.

As used in this specification, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B.

The term “about” as used herein when referring to a measurable value such an amount of weight, time, dose etc. is meant to encompass variations of ±20% or ±10%, more preferably ±5% from the specified amount as such variations are appropriate to perform the disclosed method.

The term “prolamin” as used herein are intended to refer to the group of plant storage proteins having a high proline and glutamine content. Depending on the source cereal, they have different names, wheat (gliadin and glutenin), barley (hordein), rye (secalin), corn (zein), sorghum (kafirin) and oats (avenin). They are generally soluble in 50% alcohol solutions.

The term “avenin” as used herein refers to the prolamin in oats. It is analogous to the gluten within wheat. Avenin proteins comprise a number of proteins of varying molecular weight, for example see Olin D Anderson, The Spectrum of Major Seed Storage Genes and Proteins in Oats (Avena sativa) PloS One 2014; 9(7): e83569.

The term “gluten” as used herein is intended to encompass both gliadins and glutenins or either protein alone.

By “water washing” it is meant is process in which a cereal flour is washed/hydrated with water to remove starch. The process is known commercially as the Martin process. The Martin process and similar technologies separate hydrated protein and starch particles by particle size difference. In the Martin process a large continuous protein and starch matrix or dough is mechanically developed after addition of water. The starch in this matrix is relatively free and not adherent to the protein matrix. Consequently the starch falls away with the wash fluid when the dough is conveyed with continuous kneading above a porous screen and is washed in excess water.

By “substantially wet” it is meant of sufficient moisture to achieve the stated objective, in this case extraction of prolamin proteins from cereal flour.

By “ambient temperature” it is meant a temperature of about 20-25° C., more preferably about 22° C.

### Method of the Disclosure

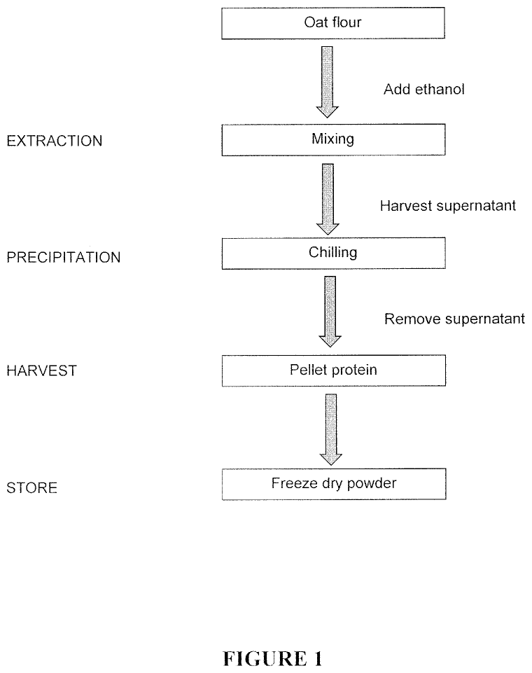

The present disclosure provides methods for purifying prolamin proteins (e.g. gluten or avenin proteins) from a cereal flour containing same. This process is depicted schematically for the preferred embodiment in FIG. 1.

The method described in detail below uses purification of avenin proteins from oat flour as an example by way of illustration.

Briefly, extraction of avenin proteins from the oat flour is achieved by soaking the oat flour in ethyl alcohol in amount sufficient to substantially wet the flour and to form an oat flour suspension. Any suitable container can be used for admixing of the flour and water e.g. bucket or beaker. There is no pre-step of mixing the oat flour with water. In certain preferred examples, the organic solvent (e.g. ethyl alcohol) is provided at a concentration within the range of about 40-60% v/v, preferably about 50% v/v. In certain examples, the ethyl alcohol is admixed with the oat flour to provide a suspension of oat flour having a concentration of at least about 2 g flour/3 mL of a 50% v/v ethyl alcohol solvent. In further examples, the oat flour and ethyl alcohol are admixed in a concentration range from about 0.2 to 2 g flour/3 mL of a 50% ethyl alcohol solvent. Mixing of the flour and ethyl alcohol can be carried out room temperature/ambient temperature. The mixing is carried out for a time sufficient to provide optimum extraction of the avenin proteins from the cereal flour. Depending on the flour content, the extraction period may last anywhere from between 10-30 mins on a laboratory scale to about 1 to 24 hours on a commercial scale. In certain examples, the extraction period is performed overnight (about 8 to 12 hours) for convenience. The mixing process may be continuous or intermittent. Various mixing apparatus for carrying out the mixing process would be familiar to those skilled in the art and include, for example vortex machines, magnetic stirring machines or motorised blenders, including industrial scale vertical blenders like the Hobart mixer.

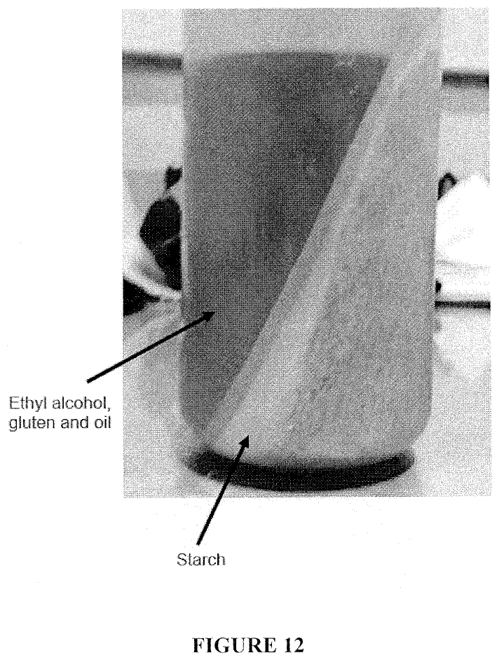

The avenins are separated from the other components in the flour (e.g. starch) by physical separation techniques such as filtration, centrifugation, decanting or settling. This is shown in FIG. 12 where the flour and starch bands form discrete bands following centrifugation. Depending on the volume it may be necessary to perform the centrifugation in batches. If centrifugation is used, a sufficient speed and time is utilised so that a firm pellet is formed (see for example, FIG. 12). Persons skilled in the art will be familiar with appropriate centrifugation speeds and times. By way of non-limiting example as described herein, a 500 mL oat flour/EtOH suspension is centrifuged at 800×g for about 5 min. In some instances, multiple rounds of centrifugation and precipitation are carried out to increase purity and recovery.

Precipitation of the avenin proteins is achieved by chilling the supernatant from the extraction step. The inventors have found that significant avenin precipitation can be achieved allowing the avenins to settle under gravity over a period of about 60 mins on a laboratory scale to about 2 days on a commercial scale. Preferred chilling temperatures are in the range of about 4-15° C., more preferably about 4-10° C. Chilling can be achieved by methods known in the art, such as refrigeration or placement of the container containing the supernatant on a refrigerated surface or atmosphere.

Harvesting the precipitated avenins may be achieved by any means known in the art including filtration, centrifugation, decanting or settling. The inventors found that avenin proteins readily settle at 4° C. under gravity and centrifugation is not necessarily required and the supernatant can simply be decanted. A milky white precipitate forms. Purification efficiency can be enhanced by centrifugation of the supernatant to recover the avenin proteins which are still remaining in solution. The pellet following centrifugation forms a clear honey-like liquid. The ethyl alcohol can be recovered for re-use.

The purified avenin proteins may be stored by various means as described herein.

For example the pellet may be resuspended in a minimal volume of water or dilute ethyl alcohol. If ethyl alcohol is used then the concentration is sufficient to prevent or delay microbial contamination. In certain examples, the ethyl alcohol concentration is at about 10% v/v. The resuspended pellet is preferably stored chilled but can also be stored in a freezer. A powdered form of the avenin proteins can be produced by evaporating the ethyl alcohol according to standard methods, for example, vacuum evaporation (frozen or freeze-drying) or evaporation in air or drying with heat. Where retention of the protein baking function and properties is desired, drying of the avenin protein fraction should be carried out at a temperature no higher than about 65° C.

In certain examples, when the avenin protein fraction has been stored in dilute ethyl alcohol it may be first necessary (prior to freeze drying) to break up clumps of avenin which can be carried out by methods known in the art e.g. blending or vortexing. The resulting powder can be stored chilled, preferably about 4° C. or it can be stored at room temperature.

By practise of the methods of the disclosure, protein yields of 70% or greater.

Additionally, by practice of the methods of the disclosure, purity yields of 90% or greater can readily be obtained.

The above process can also be utilised as described above for the extraction of purification of gluten proteins from gluten containing cereals, for example wheat, barley and rye.

The present process avoids the disadvantages associated with prior art methods. For example, many prior art methods utilise the Martin process (water washing) which requires the use of water to wash out the starch, followed by drying of the wet residue. Wet gluten is sticky and manipulating and drying industrial quantities using gentle heat to avoid damaging the baking properties of the gluten/avenin is difficult and slow. In contrast, the present process avoids the need for water washing or the addition of any water. The process can also be performed without the need for applied heat. A further advantage is that the process avoids production of large volumes of waste water, and the ethanol used can be reclaimed by evaporation and reused. Since no relaxing step of a batter or dough is required, the process is shortened.

The purity of the proteins are also substantially higher compared to proteins purified according to traditional methods.

The avenin protein fraction has altered and improved properties. It can be used alone or as a supplement to gluten free, or gluten-containing flour to provide improved dough strength and dough stability.

The protein fraction minds many uses, both food and non-food uses. It can be used as the protein fraction constituent of a gluten free or gluten containing flour composition to improve bread baking functional properties and elastic properties. Non-food uses include films, adhesives, plastics and in paper and cardboard making to stiffen them.

Admixing, precipitation and centrifugation times used in the process will depend on the quantity of flour being processed. These parameters can readily be determined by test runs and the like.

### Prolamin Proteins

There is a complex diversity in the primary structures of the gluten-like proteins which are collectively known as prolamins. Prolamins are a family of closely homologous, alcohol soluble, seed storage proteins consisting of gluten in wheat (Triticum spp. L., composed of a mixture of gliadins and glutenins), hordeins in barley (Hordeum vulgare L.), secalins in rye (Secale cereal L.), and avenins in oats (Avena sativa L.).

Unfortunately, there is not a single gluten protein; rather, wheat gluten is a complex mixture of several hundred related proteins, containing members of the monomeric α-, γ-, and ω-gliadins and the high and low molecular weight glutenins, which form polymers in vivo. The hordeins consist of four protein families: the B-, C-, D-, and γ-hordeins. The B- and C-hordeins account for 70% and 20% of the hordeins, respectively, while the D- and γ-hordeins are minor components accounting for less than 1% and 5% of total hordeins, respectively. The B- and C-hordeins are both multi-gene families with 2D protein gels showing upwards of 10 individual B- and C-hordeins. The D- and γ-hordeins are coded for by one and three genes respectively, with 2D protein gels showing approximately five D-hordein isoforms.

The secalins are also multi-gene families of four protein families with the prolamins accounting for 65% of seed protein, and within that, the γ-75k secalins accounting for about half of the prolamin, followed by γ-40k secalins (24%), the ω-secalins (17%), and HMW secalins at 7% of prolamin.

The oat avenins are multi-gene families of at least 20 proteins, with homology to the α- and γ-gliadins of wheat, the B hordeins of barley, and the γ-secalins of rye. These genes are distributed across a single chromosome and do not contain homologous sequences to the gliadin-33-mer or -17-mer; however, they do contain immunoreactive peptides QQPFVQQQQQPFVQ and QQPFMQQQQPFMQP with the repetitive epitopes PFVQQQ and PFMQQQ.

All of the above prolamins are immunoreactive with celiac T cells as they share repeated runs of amino acid sequence with other celiac immunoreactive prolamins. However, it appears that approximately 10% of celiacs have a genuine T-cell mediated reaction to avenins. The reaction of an individual celiac depends upon the concentration of immunoreactive prolamins and the degree of immunoreactivity of the prolamins. The 10% of celiacs who react to oats may represent a cohort of celiacs who are very sensitive and react to the low level of avenins found in oats or they may be subjects who can mount a T-cell response to avenins for other reasons. Thus, consumption of uncontaminated oats is suitable for most, but not all celiacs.

Prolamin proteins also occur in gluten-free grains such as such as maize (Zea mays L.), rice (Oryza sativa L.), and sorghum (Sorghum bicolor L. Moench), which are distant relatives of wheat; however, these prolamins are distantly related to the gluten proteins of wheat.

Maize prolamins (zeins) may provoke a celiac response in some celiacs, but are generally considered safe for most celiacs to consume. The prolamins from rice and sorghum do not contain homologous sequences to the 33-mer gliadins or the 17-mer-gliadin and also lack the extensive and repetitive PSQQ and QQQP epitopes present in wheat gliadins and glutenins. Rice and sorghum do not provoke a celiac response.

In rice, there are three families of prolamins (sometimes called oryzeins): the 10 kDa, 13 kDa, and 16 kDa prolamins encoded by single, multiple (up to six), and single genes, respectively. In maize, the 22 kDa and 19 kDa zeins are encoded by large multi-gene families with over 20 members. In sorghum, there are four families of kafirins: α-kafirins (the most abundant, 80-85% of total kafirin) at 23 and 25 kDa, β-kafirins (7-13%) at 19 kDa, and γ-kafirins (10-20%) at 20 kDa. A fourth group of kafirins, related to the 6-zeins of maize, has been identified from cDNA sequences (for review see Tanner et al. (2014) J. Am. Soc. Brew. Chem. 72(1):36-50).

### Isolation of Gluten and its Uses

Gluten isolation is a worldwide multibillion dollar industry, operating in USA, Australia and Europe and used to isolate many tens of million tonnes of purified “vital’ gluten. Purified gluten is required to be added back to flour doughs during the baking process to standardise dough strengths of different batches of bread wheat flour. Vital gluten is used to designate that the isolated gluten has not lost baking quality during the isolation.

Of all the cereal grains, wheat is produced in the largest tonnage around the world. Wheat is most often dry milled into farina, flour, germ and bran which are converted into food or feed. Dry milled products are mixtures of proteins, carbohydrates, lipids, phenolics and fiber. Wet processing of wheat provides end products of singular compositions such as protein (gluten), starch and oil. Gluten is traditionally isolated from fine wheat flour by washing out the water soluble components. Wheat is milled into flour, the flour is mixed into a 50% solids dough and “worked” to form the cohesive structure of gluten, the dough is then washed to remove the starch and soluble fractions, and the formed gluten is washed until the required protein content is reached, i.e. soluble proteins are removed lowering the protein content to that of purified gluten. Gluten that is intended to supplement wheat flour in the baking process is called “Vital gluten” to denote that the baking properties are retained to at least some degree. This washing process can use large volumes of water which is costly to dispose of. The sticky, wet gluten is dried by heating. Apart from the high protein content, vital wheat gluten is also rich in essential minerals such as phosphorus.

Vital wheat gluten is widely used for making seitan, a vegetarian substitute for the meat which is popular among the vegetarians and vegans. Vital wheat gluten is also used as a binder for various food products such as meatballs, meatloaf, and tofu among others, which in turn is driving the global market for vital wheat gluten.

As shown in the Examples provided herein, the inventors have shown that the process of this disclosure is also suitable for purifying gluten proteins from gluten containing cereals such a wheat, barley and rye.

In some examples, the method of the disclosure is applied to mixtures of gluten containing flours.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Methods

### Urea-SDS-PAGE

Gluten proteins were dissolved in no more than a 1:1 dilution of sample in 6M urea, 62.5 mM Tris-HCl (pH 6.8), 10% glycerol, 2% (w/v) SDS, 0.01% (w/v) Bromophenol Blue, and 65 mM DTT (freshly added) at RT. Proteins were resolved by gel electrophoresis in 1 mm Zoom 4-12% Bis-Tris polyacrylamide gels (Invitrogen) using MOPS-Tris-SDS buffer as instructed at 200V for 55 min, fixed in 40% (v/v) aqueous MeOH, 10% (v/v) glacial acetic acid, rinsed in water, stained with 0.06% (w/v) colloidal Coomassie Blue G250 in 8.5% (v/v) phosphoric acid and 10% ethanol (EtOH) and de-stained in water overnight. Gels and blots were imaged and calibrated using pre-stained protein standards (Invitrogen) using a BioRad Chemi-doc. Relative protein concentrations were deduced from the ratio of band density to total lane density.

### Protein Determination

Protein was determined by the method of Bradford (Bradford, MM (1976) Analytical Chemistry, 72, 248-254).

### Western Blot

Protein gels run as above, were blotted to nitrocellulose membranes using iBlot (Invitrogen) semi-dry blotter (program 0). Blots were blocked in fresh 5% (w/v) dry skim milk powder, 1% (v/v) Tween 20, in phosphate buffered saline (PBS) overnight at 4° C. Blots were exposed to Sigma rabbit anti-gliadin-HRP conjugate, raised to native and heat-treated wheat gliadin (Sigma A1052-1 ML) at 1/1000 (v/v) for 1 hr, washed in PBS and signal developed with Amersham ECL reagent and the image scanned (BioRAD Image Master). The Sigma-anti-gliadin_HRP antibody has been shown to be a general anti-gluten antibody, detecting all gluten proteins in wheat, barley, rye and oats (Colgrave, M. L., et al., (2015) Journal of Proteome Research, 14(6), 2659-2668).

### Effect of Solvent Polarity on Small Scale Avenin Precipitation

Oat flour (500 g) was extracted twice in 50% ethanol (EtOH) (750 mL) and the extracts pooled as below. Duplicate 10 ml aliquots of the pooled 50% EtOH extract containing 5.2 mg protein/ml was subject to varied total EtOH concentration by either diluting the 50% EtOH extract with either water, to achieved final EtOH concentrations of 10-41%, or with EtOH to achieve final EtOH concentrations of 66%-90%. In addition the 50% extract was chilled at 4° C. and centrifuged as below. Solutions were centrifuged at 5000 g/10 min/RT and pellets were dissolved in 8M urea, 1% (w/v) DTT, 20 mM triethylamine-HCl (pH 6) overnight, the protein content measured and subject to Urea-SDS-PAGE and western blot as above.

### Five Hundred Gram Oat Extract

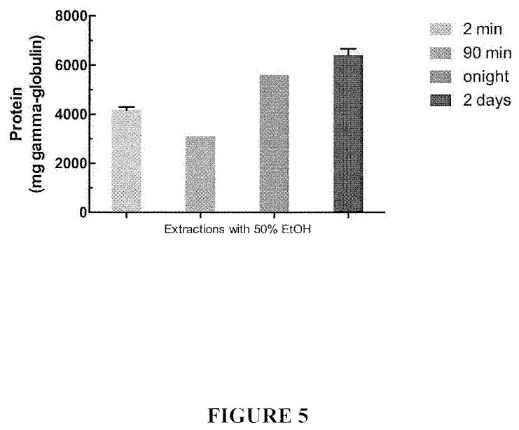

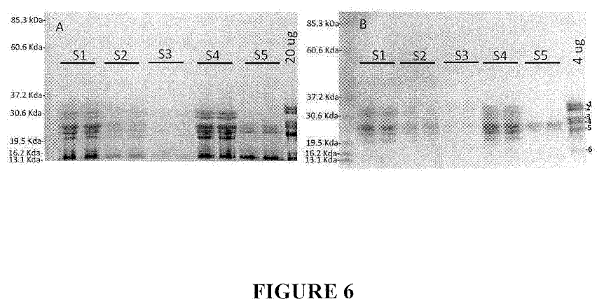

Oat flour (500 g) was shaken occasionally over 2 min, 90 min, or overnight for one or 2 days in 750 mL of 50% (v/v) EtOH and then centrifuged at 500 g and supernatants pooled (S1). Pellets were resuspended in 750 mL 50% (v/v) EtOH and re-centrifuged and the process repeated (S2, and S3). The pooled extracts were combined (S4) and chilled at 4° C./10 min and centrifuged at 5,000 g at 4° C. to yield a final avenin pellet and a supernatant (S5). The total yield of protein for each extraction method, was calculated from the protein content of the respective combined supernatants (FIG. 5) measured by Coomassie Blue. The avenin pellet was resuspended in 100 mL of 10% (v/v) ethanol at 20° C., and freeze dried. A portion of the freeze dried avenin was dissolved in 8M urea, 1% DTT, 20 mM Triethylamine-HCl (pH 6) at 1.0 mg/ml and either 20 μg or 4 ug applied to a single lane of a protein gel (FIG. 6A, 20 μg) or western blot (FIG. 6B, 4 μg) respectively. Total protein was determined in each fraction, calibrated against gamma-globulin using Coomassie Blue. Coomassie stained protein gel (FIG. 6A) and Sigma-anti-gliadin antibody stained western blot (FIG. 6B) was used to examine the recovery and purity of avenin.

### Large Scale 200 kg Sequential Oat Extraction

The above method was scaled-up to generate a larger scale avenin isolation method capable of extracting 200 kg of oat flour.

Two crops of wheat free oats were grown in the Williams Shire, in the south-east of West Australia, using dedicated wheat free machinery and cropland and 200 kg lots transported to Melbourne in sealed “bulka” bags. The crops were harvested in December 2016 and 2017. Prior to grinding of each crop, sequential lots of 12×100 g of oats was spread thinly on a tray and examined for other grains. No wheat, barely or rye-grass was detected in any samples. No other material was detected—confirming the purity of the wheat free oats. The oat grain was ground to pass a 40 hole/in screen, in a dedicated gluten-free hammer mill kindly supplied by Wards Mackensie (Altona, Australia). The flour was captured in eight 25 kg bags. Before each bag was sealed, a 100 g flour sample was taken from each bag for testing for accidental contamination by herbicides and pesticides. For each crop, eight successive flour samples were screened for a panel of 200 herbicides and pesticides, and six common aflatoxins by Agrifood Technology, Werribee, Vic. Ten (10) g of each purified avenin was also screened for heavy metal contamination. No inadvertent chemical, heavy metal or biological contamination was detected. The flour was extracted using food grade procedures and EtOH in a lab decontaminated to remove traces of wheat flour (Manildra Group, Nowra). All containers used for solvent storage and extraction were Food and Drug Approved and BPA free (Bunnings, Australia).

Over the course of 9 days in January 2018, and May 2018, 200 kg of fine wheat-free, oat flour was subject to additional grinding in a blender, and extracted with 50% (v/v) EtOH as follows. First 8 kg lots of oat flour were soaked in 12 L of 50% (v/v) EtOH overnight with occasional mixing at room temp. In the morning, the oat suspension was stirred and decanted into successive 6×500 ml buckets and centrifuged at 800 g/5 min in a Sigma 6-16S centrifuge at ambient temperature to give a firm pellet. FIG. 12 shows the pellet formed with the starch band and flour bands clearly visible after centrifugation. The oil, ethanol and avenin proteins are present in the supernatant fraction.

The clear supernatants were pooled in a 30 L bottle and chilled at 4° C. for 2 days to selectively precipitate the avenins. Significant avenin settled after 2 days storage at 4° C., and was removed from the bottom of the storage container as below. The avenin precipitate which remained in suspension, was collected by centrifugation at 5,000 g/10 min at a chilled temperature and formed a clear honey-like liquid which was resuspended in a minimum volume of 10% (v/v) EtOH, and stored at 4° C. Clumps of precipitated avenin were dispersed with an overhead blender, frozen and freeze dried in a dedicated facility according to standard methods to yield a white powder which was stored dry at 4° C. until required. Final yields of freeze dried avenin were 1.2 and 0.9 kg for preps 1 and 2, respectively.

A Generalised Gluten Isolation Method from Oats, Barley and Wheat by Chill Precipitation

Chill precipitation was shown to be applicable as a general gluten isolation method. Five gm of fine flours from wheat, barley and oats were extracted in 15 mL of 50% (v/v) EtOH, by vortexing regularly over a period of 1 hr at RT and centrifuged at 3,200 g/1 hr. The clear supernatants were chilled at 4° C. and within a few minutes cloudy precipitates had formed. These were collected by centrifugation at 3,200 g/5 min and the pellets of wheat, barley and oats dissolved in 8M urea, 1% DTT, 20 mM TEA (pH 6), 10 mL, 10 mL, or 1 mL respectively. The proteins in these chill precipitated preparations were compared to those present in freshly isolated gluten extracts isolated by extracting wheat (50 mg), barley (50 mg) and oats (100 mg) flour by vortexing in 1 mL of 50% IPA, 1% DTT in duplicate. Protein was measured and indicated protein levels was loaded on SDS-PAGE protein gels.

### Results

### Example 1 Effect of Solvent Polarity on Avenin Precipitation

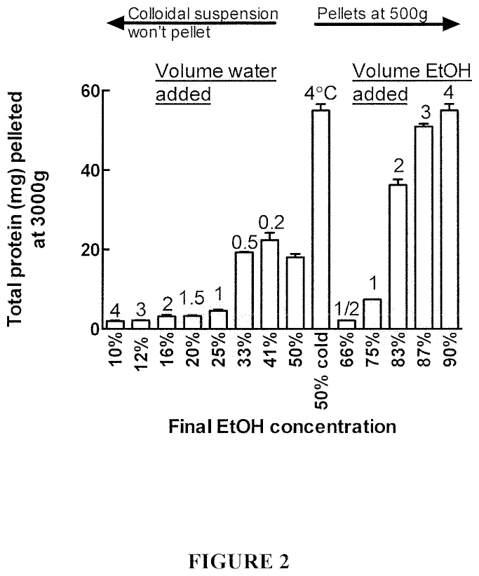

Most gluten proteins can be dissolved in 50% (v/v) ethanol (EtOH) or propanol and precipitated by dilution with either water or alcohol. However under some circumstances, precipitated oat avenin resisted centrifugation. The polarity of the 50% EtOH avenin extract was varied by either diluting the 50% EtOH extract either with water, to achieved final EtOH concentrations of 10-41% (v/v), or with EtOH to achieve final EtOH concentrations of 66-90 (v/v) (FIG. 2).

All additions (water or ethanol) to the 50% EtOH extract produced a milky white precipitate—however only those precipitates produced by increasing the EtOH addition could be spun down at 3,000 g. The cloudy precipitate produced by adding water was extremely difficult to spin down. Avenin precipitates induced by lowering the EtOH concentration with water resisted centrifugation. Fortuitously, the inventors discovered that chilling the 50% EtOH extract at 4° C./10 min selectively precipitated avenin, producing a milky white precipitate that could spun down at either 500 g or 3,000 g/10 min (FIG. 2) or settled at 1 g overnight. However, chilling the water-induced precipitates at 4° C. did not help the water suspensions to precipitate. The inventors postulate that the need to sediment the 4° C. precipitate at 5,000 g may be a result of lipid binding to the avenin and “floating” it. Defatting with butanol, ether or hexane is not possible due to food safety concerns—e.g. hexane is biodegraded to form a cumulative neurotoxin and must be avoided to maintain food grade standard. The chilling induced precipitation of avenin commenced below 17° C. and could be reversed by warming, resulting in a clear solution.

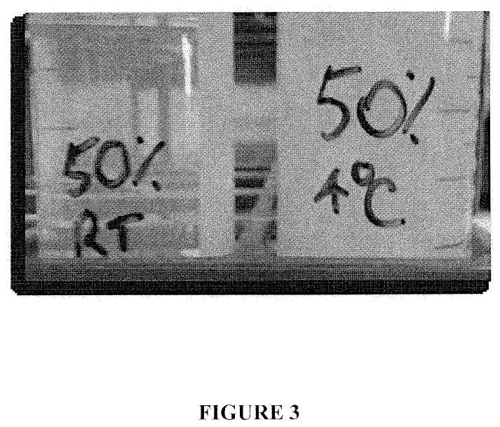

The precipitation was reversible and the chilling/warming could be repeated at least 10 times (FIG. 3). Successful and complete defatting of oat flour with 100% EtOH has been reported (Zhou, Robards, Glennie-Holmes, & Helliwell, (1999) Journal of Agriculture and Food Chemistry 47(10):3941-53). However EtOH defatting did not reduce the g forces required to pellet the 4C pellet from the chilled 50% EtOH extract.

### Example 2 Purity of Avenins

The avenins can be seen in the western blot visualised with Sigma rabbit anti-gliadin-HRP conjugate, raised to native and heat-treated wheat gliadin (Sigma A1052-1 ML). The Sigma antibody has previously been shown as suitable as a general antibody to visualise gluten proteins including avenins (Colgrave, M. L., et al., (2015) Journal of Proteome Research, 14(6), 2659-2668).

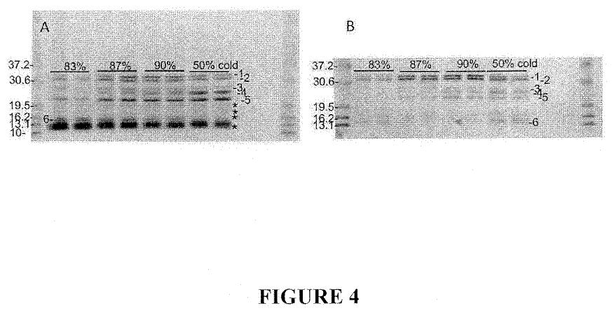

The avenins appeared as a doublet at 33.0, 31.5 kDa (see FIGS. 4A—Protein gel and B—western blot bands 1 and 2), a triplet at about 28.6, 27.3, 25.3 kDa (FIGS. 4A and B, bands 3, 4 & 5) and a band at about 16 kDa (FIGS. 4A and B band 6). These molecular weights resemble those previously observed for avenins (11.58, 22.38, 30.87, 31.50, but missing the reported 43.42 kDa (Colgrave, supra). In the western blot as the % alcohol is increased the proportion of avenin to total protein increased. These western bands corresponded to strong protein bands at the same molecular weight in the protein gel. In addition there were three faint protein bands (*) and a strong protein band below band 6 (marked with an *) not due to avenins. This indicated that at this stage the purity of the avenins was already high. The final protein contamination was less than 4%.

### Example 3 Medium Scale 500 gm Avenin Extract

Protein yield was measured with Coomassie and total protein content of each fraction was calculated. The protein recovery in the fractions was quantitative. Avenin yield was maximised by extraction for 2 days (FIG. 5). The yield of freeze dried avenin, increased from 1.4 g in the 2 min extraction to 2.0 g and 3.72 g in the overnight and 2 day extraction respectively (FIG. 5). This corresponds to a predicted maximum yield of 6.0 gm of protein, predicted by wet chemistry (Coomassie Blue calibrated with gamma-globulin). Avenin was 10% more reactive to Coomassie Blue than the standard gamma-globulin, so the wet protein determination will underestimate the true avenin level. However of the wet protein in S4 (combined supernatant) measured by Coomassie Blue, 62% was recovered as weighed freeze dried avenin. Slight losses due to incomplete precipitation and underestimation by Coomassie are most likely responsible for the short-fall.

Protein purity was determined by urea-SDS-PAGE (FIG. 6A) and Western Blot (FIG. 6B). The avenin preparation was largely uncontaminated, as shown by urea-SDS-PAGE (FIG. 6A) and western blot (FIG. 6B).

The avenin content of these fractions was estimated by western blot with Sigma rabbit anti-gliadin-HRP. Total protein content was estimated by urea-SDS-PAGE. Avenin bands 1-6 were detected as before (compare with FIG. 4). The 20 μg avenin standard (FIG. 6A, 20 μg) ran slower than expected. Gel and blots were loaded for constant volume relative to S1 (first supernatant)—i.e. if a band in subsequent lanes is half the intensity of that in S1, then the total protein yield in that fraction is also one half of that in S1. The majority of the avenin was recovered in the first 50% EtOH extract (FIGS. 6A & B, lane S1) and recovered again in the combined supernatants (FIGS. 6A & B, lane S4). Only a small amount of avenin remained in the supernatant after chilling (FIG. 6B, lane S5). Considerable non-avenin protein running at 13 kDa was recovered in the 4° C. supernatant (FIG. 6A, lane S5) indicating the majority of avenin was sedimented from the 4° C. solution.

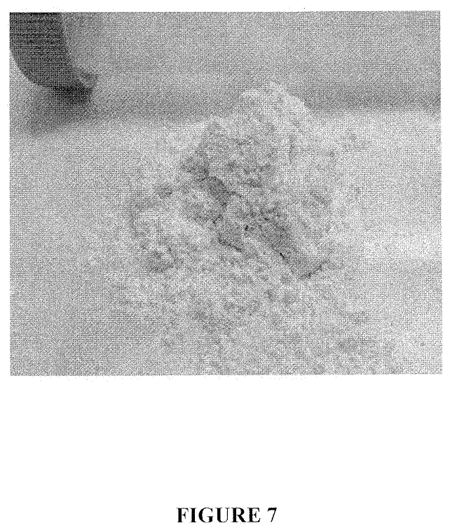

The avenins were most conveniently captured by resuspending the 4° C. pellet in distilled water (prior washing of oat flour with 1 M salt, had no effect on final avenin purity). This gave a clear white suspension of precipitated avenin which could be freeze dried to give a friable white powder (FIG. 7). In the 2 day extract, 3.72 g of freeze dried avenin was recovered from 500 g flour; i.e. 0.74% close to the expected 1%.

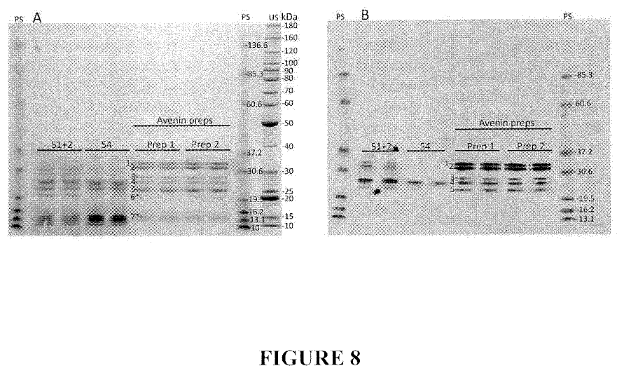

Duplicate 500 gm preparations were compared and the size of avenin bands on western blot (FIG. 8B) were compared to the size of protein bands on a protein gel (FIG. 8A). Both gel and western blot were calibrated with Invitrogen prestained standards (FIGS. 8A & B, PS), which were in turn calibrated with Invitrogen unstained 10 kDa protein ladder (FIGS. 8A & B, US). This was necessary as the prestained standards consist of proteins labelled with a dye molecule which distorts the molecular weight. The unstained protein standards consist of genetically engineered proteins which are designed to cleave into accurate protein bands. In this example, the protein content of combined supernatants from the first preparation (S1+S2) were compared to the content of the 4C chilled supernatant (S4) also from the first preparation. The protein content of duplicate freeze dried preps. is shown as FIGS. 8A & B, Prep1 and Prep2. Equal protein loads of either 20 μg (Protein gel FIG. 8A) or 2 μg (Western blot FIG. 8B) were loaded per lane. Avenin bands are shown as 1, 2, 3, 4, 5 on both the gel and blot. Contaminating proteins are shown as 6* and 7* on the protein gel. The lower avenin 16 kDa band seen in other gels is missing from the final prep but could be detected on longer processing time. Avenins were previously identified as avenin-3 (22.38, 43.42, 31.50, 30.87, and 11.58 kDa) by targeted MSMS of gel sections (Colgrave, (2015) supra).

Using the calibrated pre-stained standards on the Western blot and protein gels (FIG. 8), avenin bands were identified and the bands on protein gel or western blot were shown to be due to the same molecular weight proteins (Table 1). Avenin bands were present in the crude 50% EtOH extract (S1+2), absent from the 4° C. supernatant (FIG. 8B, S4) and enriched and relatively pure in the successive avenin preparations judged from the proportions in the protein gel.

TABLE 1
Comparison of avenin molecular weights calculated from
the protein gel (SDS-PAGE) compared to Western Blot

| Avenin | Molecular weight (western blot) | Molecular weight (gel) |
|---|---|---|
| band | kDa ± SE | kDa ± SE |
| 1 | 32.6 ± 0.02 | 33.04 ± 0.02 |
| 2 | 31.5 ± 0.02 | 31.5 ± 0.03 |
| 3 | 28.1 ± 0.07 | 28.6 ± 0.02 |
| 4 | 27.3 ± 0.08 | 27.3 ± 0.02 |
| 5 | 25.3 ± 0.09 | 25.3 ± 0.04 |

Molecular weights shown as mean +/− standard error (SE) calculated from the avenin preparations in FIG. 8.

It is clear that the western bands were due to the dominant avenin bands on the protein gel. The purity was calculated from the % of the protein load attributed to avenin bands in the protein gel. Average purity of the four lanes on the protein gel was 95.8±0.01%.

It is possible to produce a 500 g scale avenin extraction, of 96% purity from oat flour with about 60% final yield. The final avenin prep. contains five avenin bands present in the crude 50% EtOH extract (however the 16 kDa avenin also seen in the crude protein extracts was not seen in the purified prep).

### Example 4 Large Scale Avenin Purification

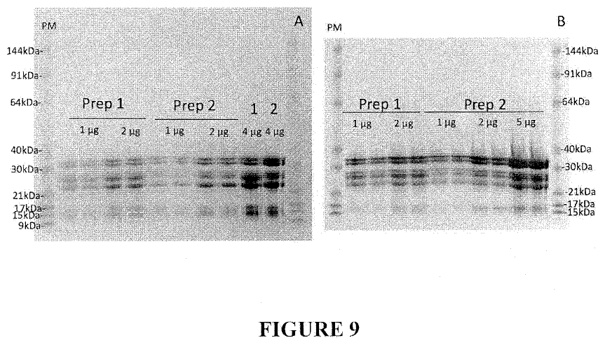

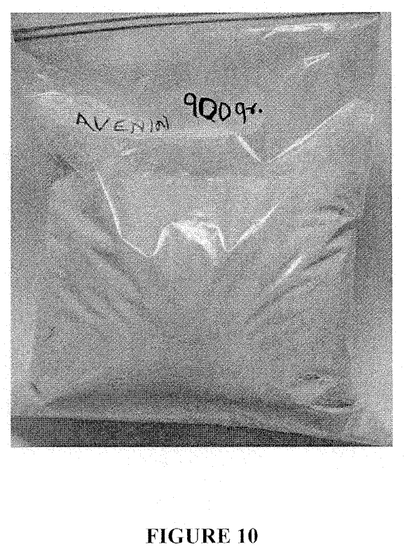

Successive 200 kg lots of oat flour were extracted, with successive 500 gm extractions as above, and the avenin chill precipitated, collected, resuspended in 10% (v/v) ethanol, and freeze dried to yield two lots of purified avenin, of 1.2 kg and 0.9 kg respectively (FIG. 10). Avenin remained as an insoluble precipitate in either water or 10% (v/v) EtOH, however 10% EtOH was used for large scale preparations to prevent microbial growth. The purity of each avenin preparation was examined by SDS-PAGE and western blotting (FIGS. 9A, B). The purity of each preparation was calculated from the summed intensity of protein bands on the protein gel, that corresponded to avenin bands on a western blot. The percentage protein purity of avenin in prep 1 was 93.01±0.36, and prep 2 was 91.93±0.16%, giving a mean purity of 92.5±0.3% over both 200 kg preparations,

### Example 5 Gluten Isolation Method

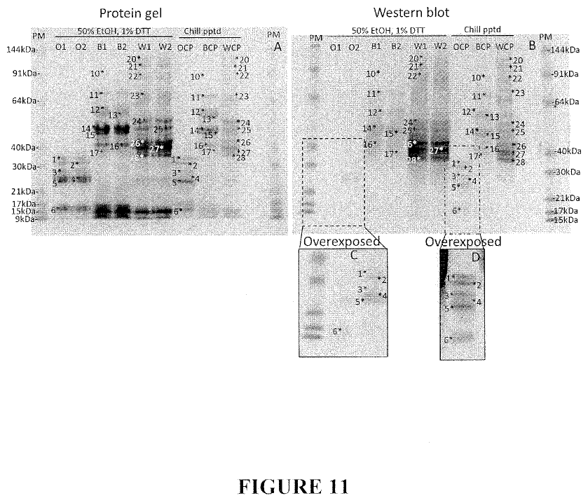

The present methods can be utilised for the purification of gluten proteins from gluten containing cereals. In every case, the gluten proteins isolated by chill precipitation and identified by Western blot (FIG. 11) by anti-gliadin antibodies (Sigma) correspond to authentic gluten proteins by definition extracted using tradition methods of 50% IPA-DTT. The Sigma anti-gliadin antibody has previously been shown to be specific for gluten proteins in a variety of grains (Colgrave et al. (2015) supra).

1. A method for purifying prolamin proteins from a cereal flour comprising said proteins, wherein the method is performed without hydrating the cereal flour with water.

2. The method according to claim 1 comprising:
(i) mixing the cereal flour with an organic solvent in an amount sufficient to substantially wet the flour and form an admixture with the flour;
(ii) chilling a supernatant for a time sufficient for the prolamin proteins to precipitate, wherein the supernatant is obtained by physically separating the cereal flour and solvent admixture of step (i); and
(iii) harvesting the precipitated prolamin proteins from the chilled supernatant of step (ii).

3. The method according to claim 1, wherein the cereal flour is selected from the group consisting of wheat flour, barley flour, rye flour, maize flour, rice flour, corn flour, sorghum flour and oat flour.

4. The method according to claim 1, wherein the prolamin proteins are selected from the group consisting of gluten proteins from wheat, secalin proteins from rye, hordein proteins from barley, avenin proteins from oats, zein proteins from maize and kafirin proteins from sorghum or combinations of one or more thereof.

5. The method according to claim 4, wherein the prolamin proteins are avenin proteins.

6. The method according to claim 2, wherein the organic solvent is selected from ethyl alcohol (ethanol), isopropyl alcohol, methyl alcohol, acetone, propanol, dimethylsulfoxide (DMSO), or dimethylformamide (DMF).

7. The method according to claim 6, wherein the solvent is ethyl alcohol (ethanol).

8. The method according to claim 2, wherein the supernatant in step (ii) is obtained by centrifugation of the admixture of step (i).

9. The method according to claim 2, wherein chilling is performed at a temperature between 4 and 10° C.

10. The method according to claim 2, wherein harvesting the precipitated prolamin proteins comprises concentrating the proteins by centrifugation to produce purified prolamin proteins.

11. The method according to claim 10, wherein the method further comprises producing a powder of purified prolamin proteins by:
(i) optionally homogenising the purified prolamin proteins; and
(ii) evaporating the water or alcohol.

12. A composition comprising substantially purified prolamin proteins prepared by the method according to claim 1.

13. A composition comprising substantially purified prolamin proteins prepared by the method according to claim 2.

14. A composition comprising prolamin proteins having a purity greater than 90%.

15. A food product or additive comprising:
(i) substantially pure avenin proteins from oat flour;
(ii) substantially pure gluten proteins from wheat flour;
(iii) substantially pure secalin proteins from rye flour;
(iv) substantially pure hordein proteins from barley flour;
(v) substantially pure zein proteins form maize flour; or
(vi) substantially pure kafirin proteins from sorghum,
wherein the food product or additive is produced by the method according to claim 1.

16. Use of substantially purified prolamin proteins prepared according to the method of claim 1 for improving dough strength and elasticity.

17. Use of substantially purified prolamin proteins prepared according to the method of claim 2 for improving dough strength and elasticity.
